(19) United States
(12) Patent Application Publication
(10) Pub. No.: US 2011/0151525 A1
(43) Pub. Date: Jun. 23, 2011

(54) ENZYMATIC PRODUCTION OF UNSATURATED FATTY ACIDS ESTERS OF ASCORBIC ACID IN SOLVENT-FREE SYSTEM

(76) Inventor: Muhammad KAIYAL, Gdeide-Maker (IL)

(21) Appl. No.: 12/972,657

(22) Filed: Dec. 20, 2010

(30) Foreign Application Priority Data
Dec. 23, 2009 (IL) 202912

(51) Int. Cl. C12P 7/64

(52) U.S. Cl. 435/134

(57) ABSTRACT

The invention provides a cost-effective enzymatic process for the production of unsaturated FA esters of ascorbic acid, such as ascorbyl oleate or ascorbyl linoleate (E-304), in solvent-free system in which ascorbic acid esters of saturated FA, unsaturated free fatty acids and/or unsaturated fatty acid esters are used as feedstock. With this method pure products can even be achieved in a short response times. These products meet the needs of the industry by improving their solubility in oil and the increased antioxidant activity, especially in foods, cosmetics and medicine.

### CROSS-REFERENCE TO RELATED APPLICATIONS

The present application claims the benefit of priority to Israeli Patent Application Serial Number 202912, filed Dec. 23, 2006, entitled “ENZYMATIC PRODUCTION OF UNSATURATED FA ESTERS OF ASCORBIC ACID IN SOLVENT-FREE SYSTEM”; The contents of the aforementioned application is incorporated herein by reference.

### TECHNICAL FIELD

In general, the present invention pertains to the arts of enzymatic reactions. In particular, the invention relates to processes of enzymatic production of unsaturated fatty acids esters of ascorbic acid.

### BACKGROUND ART

Ascorbic acid, also known as vitamin C, acts only in aqueous media as anti-oxidant, which protects the cells of the organism and the food against injury from exposure to oxygen. Their effect depends on the ability to terminate interactions with oxygen. The rancidity of oils and fats and their valuable ingredients such as color and flavor is avoided by using these anti oxidants. Because of this fundamental importance, there is a need to preserve the by oxidation permanently decreasing content of vitamin C in food during prolonged periods of storage and shelf life. Recently, several attempts were done to activate ascorbic acid in non-aquatic mediums (i.e. oil and fat) through its combination with fatty acids and producing ascorbyl esters, which should be easily soluble in oil.

These attempts are divided into two methods:

I) The chemical methods;

II) The enzymatic methods.

The chemical method succeeded only in producing saturated FA esters of ascorbic acid. The reaction takes place under extreme conditions, such as sulfuric acid or hydrogen fluoride, see for example DE 27 43 526 A1, DE 33 08 922 A1 or DE 28 54 353 C3. For this reason the production of unsaturated fatty acid esters of ascorbic acid is impossible. Furthermore products which are prepared by these processes have an odor which is not accepted by the user even after customary working (patent-Gruning, Burghard).

The second method is the enzymatic method. All previous enzymatic methods have used ascorbic acid as an acceptor alkyl. The insolubility of ascorbic acid in organic solvents slowed the process and made it expensive. Such processes have not achieved remarkable success; therefore it is not suitable for commercial large-scale manufacturing applications.

### SUMMARY OF THE INVENTION

According to the above described state of the art, the present invention seeks to realize the following objects and advantages:
1) Pure Products of unsaturated FA esters of ascorbic acid.
2) Preparation of suitable antioxidants for fish oil, such as. EPA and DHA ascorbyl esters.
3) Short Response Times.
4) Cost-effective production.
5) Prevention of turbidity, precipitation by their higher solubility in oil and Fat.
6) Higher solubility of the product in other antioxidants such as tocopherols.
7) Long shelf life.
8) Hugely reduced amounts of Antioxidants in foods.
9) Remove unwanted odors.
10) Producible as dragées.

### DESCRIPTION

The present provides an improved enzymatic process for the production of unsaturated fatty acid esters of ascorbic acid. Below the general flow of process of the invention is illustrated.

### FIRST EMBODIMENT

Saturated fatty acids esters of ascorbic acid are reacted with unsaturated free fatty acids in the presnce of preferibly emobolized enzyme. The aformentioned reaction results with unsaturated fatty acids esters of ascorbic acid and saturated free fatty acids.

### SECOND EMBODIMENT

Saturated fatty acids esters of ascorbic acid are reacted with unsaturated fatty acid esters in the presnce of preferibly emobolized enzyme. The aformentioned reaction results with unsaturated fatty acids esters of ascorbic acid and saturated fatty acid esters.

### WORKING EXAMPLES

The conversion of the raw materials was determined by measuring the percentage of unsaturated fatty acid esters of ascorbic acid and saturated fatty acid esters of ascorbic acid using gas chromatography (GC) after 3 hours of reaction time. Measurements: The measurements were performed using gas chromatography, HP 5800, 30 meter Quadrex 5% phenyl 95% methyl silicone capillary column, column temperatures: 250C degree to 320C degree, R 5C degree/min.

### References of Materials:

1) Oleic acid 95%, sigma, Cat Nr. O1630
2) Oleic acid ethyl ester 98%, sigma, Cat Nr. O9500
3) Linoleic acid 95%, sigma, Cat Nr L1268.
4) Ascorbyl palmitate (6-O-Palmitoyl-L-ascorbic acid) 99%, sigma, Product Nr 76183.
5) Lipozyme TL-IM, (Lipase from Thermomyces lanuginosus), Novozymes.
6) Novozym 435 (Lipase from Candida Antarctica), Novozymes.
7) Lipozyme RM IM (Lipase Rhizomucor miehei).

### Example 1

Enzymatic production of ascorbyl-oleate with the use of ascorbyl-palmitate and oleic acid as raw materials.

The enzymatic reaction was carried out by adding immobilized enzyme, Novozym RTM 435 (5 g) into double-jacketed glass reactor, which contains the starting materials 6-ascorbylpalmitate (50 g) and oleic acid (136.4 g), (a stoichiometric ratio of 1:4 between 6-ascorbyl palmitate and oleic acid, respectively). The reaction mixture was mechanically stirred (250 rpm) at 45<0>C for 3 hours. The excess of the oleic acid was removed by using distillation procedure. After 3 hours stirring was 92% of the saturated fatty acids of ascorbic acid in unsaturated fatty acid of ascorbic acid converted.

### Example 2

Enzymatic production of a mixture of unsaturated fatty acid ester of ascorbic acid with the use of ascorbyl palmitate and oil as a feedstock.

The enzymatic reaction was carried out by adding immobilized enzyme, Novozym RTM 435 (10 g) into double-jacketed glass reactor, which contains the starting materials 6-ascorbylpalmitate (50 g) and soybean oil (135.2 g), (a stoichiometric ratio of 1:4 between 6-ascorbyl palmitate and soybean oil, respectively). The reaction mixture was mechanically stirred (250 rpm) at 45<0>C for 3 hours. The excess of the oil was removed by using distillation procedure. After 3 hours stirring was 85% of the saturated fatty acids of ascorbic acid in unsaturated fatty acid of ascorbic acid converted.

### Example 3

Enzymatic production of a mixture of unsaturated fatty acid ester of ascorbic acid with the use of ascorbyl palmitate and oil as a feedstock.

The enzymatic reaction was carried out by adding of the immobilized catalyst, Lipozyme .RT IM (10 g), to ascorbyl palmitate (50 g) and soybean oil (135.2 g) (a stoichiometric ratio of 1:4 between 6-ascorbyl palmitate and oil, respectively) into double-jacketed glass reactor. The reaction mixture was mechanically stirred (250 rpm) at 45<0>C for 3 hours. The excess of the soybean oil was removed by using distillation procedure. After 3 hours stirring the conversion was 82%.

### Example 4

Enzymatic production of ascorbyl-oleate with the use of ascorbyl-palmitate and oleic acid ethyl ester as raw materials.

The enzymatic reaction was carried out by adding immobilized enzyme, Lipozyme RM IM (10 g) into double-jacketed glass reactor, which contains the starting materials 6-ascorbylpalmitate (50 g) and oleic acid ethyl ester (136.4 g), (a stoichiometric ratio of 1:4 between 6-ascorbyl palmitate and oleic acid ethyl ester, respectively). The reaction mixture was mechanically stirred (250 rpm) at 45<0>C for 3 hours. The excess of the oleic acid ethyl ester was removed by using distillation procedure. The conversion was 88%.

### Example 5

Enzymatic production of ascorbyl linoleate using 6-ascorbyl palmitate and linoleic acid as a reaction start material.

The reaction was carried out by adding of the immobilized catalyst, Lipozyme TL-IM (10 g) to 6-ascorbyl palmitate (50 g) and linoleic acid (138.5 g) (a stoichiometric ratio of 1:4 between 6-ascorbyl palmitate and linoleic acid, respectively) into double-jacketed glass reactor. The reaction mixture was mechanically stirred (250 rpm) at 45<0>C for 3 hours. The excess of the linoleic acid was removed by using distillation procedure. The conversion was 95%.

### Example 6

Enzymatic production of a mixture of unsaturated fatty acid ester of ascorbic acid with the use of ascorbyl palmitate and oleic acid and linoleic as a feedstock.

The reaction was carried out by adding of the immobilized catalyst Lipozyme TL-IM (10 g) to 6-ascorbyl palmitate (50 g) and oleic acid (68.2 g) and linoleic acid (69.25 g) (a stoichiometric ratio of 1:2:2 between 6-ascorbyl palmitate and oleic acid and linoleic acid, respectively) into double-jacketed glass reactor. The reaction mixture was mechanically stirred (250 rpm) at 45<0>C for 3 hours. The excess of the oleic acid and linoleic acid was removed by using distillation procedure. After 3 hours stirring was 85% of the saturated fatty acids of ascorbic acid in unsaturated fatty acid of ascorbic acid converted.

1. A process for an enzymatic conversion of saturated fatty acids esters of ascorbic acid into the unsaturated fatty acids esters of ascorbic acid, esseensailly as described herein.

2. The process according to claim 1, wherein said saturated fatty acids ester of ascorbic acid and unsaturated free fatty acid are the starting materials.

3. The process according to claim 1, wherein said saturated fatty acids ester of ascorbic acid and unsaturated fatty acid ester are the starting materials.

4. The process according to claim 1, wherein at least one saturated fatty acids esters of ascorbic acid reacts with at least one unsaturated free fatty acids and/or reacts with at least one unsaturated fatty acid esters.

5. The process according to claim 1, wherein at least one saturated fatty acids esters of ascorbic acid is reacting with mix of unsaturated free fatty acid and unsaturated fatty acid ester.

6. The process according to claim 1, wherein said reaction is performed within a medium without solvent.

7. The process according to claim 1, wherein that the used immobilized enzymes, selected from lipases, esterases or proteases.

8. The process according to claim 1, wherein at least one immobilized enzyme is used.

9. The process according to claim 8, wherein said enzyme is immobilized on an insoluble carrier.
